(19) United States
(12) Patent Application Publication
(10) Pub. No.: US 2019/0298175 A1
(43) Pub. Date: Oct. 3, 2019

(54) BIOLOGICAL INFORMATION MEASUREMENT APPARATUS AND BIOLOGICAL INFORMATION MEASUREMENT METHOD

(71) Applicant: CANON KABUSHIKI KAISHA, Tokyo (JP)

(72) Inventors: Norio Matsui, Mishima-shi (JP); Daisuke Kaneko, Suntou-gun (JP)

(21) Appl. No.: 16/364,255

(22) Filed: Mar. 26, 2019

(30) Foreign Application Priority Data
Mar. 27, 2018 (JP) 2018-060730

(51) Int. Cl. A61B 5/00

(52) U.S. Cl. CPC A61B 5/0075; A61B 2090/306

(57) ABSTRACT

A biological information measuring apparatus comprises: a light source; a guide unit that guides a luminous flux from the light source to a measurement target, which is part of a living body, and guides reflected light from the measurement target; a spectral unit that disperses the reflected light guided by the guide unit, with a predetermined resolution in a predetermined wavelength range; and an obtaining unit that sequentially obtains light intensities for a predetermined wavelength, which is part of light of a plurality of wavelengths obtained by the spectral unit.

### BACKGROUND OF THE INVENTION

### Field of the Invention

The present invention relates to a biological information measurement apparatus and a biological information measurement method for measuring biological information by illuminating a living body with light and detecting temporal variation in the light amount of the reflected light or transmitted light therefrom.

### Description of the Related Art

In recent years, a vital sensor has been commercially available which illuminates part of a human body with light having a specific wavelength and detects a blood pulse wave (hereinafter referred to as a pulse wave) accompanying the movement of blood by using a light receiving sensor to detect the amount of reflected light or the amount of transmitted light from blood moving through blood vessels in a living body. The pulse wave is used to measure the pulse rate. Also, it has been proposed that a degree of blood vessel stiffness caused by aging of the blood vessel inner wall or accumulated matter is obtained using the acceleration pulse wave obtained by finding the second-order differential of the pulse wave, and this degree of blood vessel stiffness is presented as the blood vessel aging degree or the blood vessel age.

In general, this type of measurement apparatus illuminates the measurement target with light using a light source formed by an LED having a specific wavelength, the reflected light or transmitted light from the measurement target is detected by a light receiving sensor, and the amount of temporal variation in the output is measured. Specifically, a target portion, such as a fingertip, a wrist, or an ear lobe, is illuminated with light using an LED light source having a green or red wavelength, and the temporal moving state of the blood in the blood vessels, that is, the pulse (pulse wave), is detected based on variation in the amount of light that is reflected from or passes through the target portion. Japanese Patent Laid-Open No. 2004-000467 discloses a pulse wave measuring apparatus that illuminates a fingertip with a luminous flux emitted by a light source, receives the reflected light therefrom with a photoelectrical conversion light receiving sensor, and measures and evaluates the temporal variation in the received light amount.

However, in the light emitted from the LED being used as the above-described light source, although light with a predetermined wavelength is dominant, light of other wavelengths is also included, which results in noise caused by light with unneeded wavelength components. Also, due to light rays from the outside entering the light receiving sensor in combination with the reflected or transmitted light from the target portion, unneeded wavelength components have been added to the light reception signal as noise.

### SUMMARY OF THE INVENTION

The present invention provides a biological information measurement apparatus that can measure biological information stably and with high accuracy.

According to one aspect of the present invention, there is provided a biological information measuring apparatus, comprising: a light source; a guide unit configured to guide a luminous flux from the light source to a measurement target, which is part of a living body, and to guide reflected light from the measurement target; a spectral unit configured to disperse the reflected light guided by the guide unit, with a predetermined resolution in a predetermined wavelength range; and an obtaining unit configured to sequentially obtain light intensities for a predetermined wavelength, which is part of light of a plurality of wavelengths obtained by the spectral unit.

According to another aspect of the present invention, there is provided a biological information measurement method performed by a biological information measurement apparatus, the method comprising: guiding a luminous flux from a light source to a measurement target, which is part of a living body; dispersing reflected light from the measurement target with a predetermined resolution in a predetermined wavelength range by guiding the reflected light to a spectral unit; and sequentially obtaining light intensities for a predetermined wavelength, which is part of light of a plurality of wavelengths obtained through the dispersion performed by the spectral unit.

Further features of the present invention will become apparent from the following description of exemplary embodiments with reference to the attached drawings.

### BRIEF DESCRIPTION OF THE DRAWINGS

FIGS. 1A to 1C are external perspective views showing a pulse wave measuring apparatus according to a first embodiment.

FIGS. 2A to 2C are diagrams illustrating a structure of a spectrometer.

FIG. 3 is a perspective diagram illustrating an optical path of the spectrometer.

FIG. 4A is a top view illustrating an optical system of the spectrometer.

FIG. 4B is a perspective diagram illustrating an optical system of a spectrometer.

FIG. 5A is a diagram showing an optical absorption property of hemoglobin.

FIG. 5B is a diagram showing an example of results of measuring output amplitudes at all wavelengths.

FIG. 5C is a diagram showing an output (pulse wave) of light intensity for a specific wavelength.

FIG. 6 is a diagram illustrating a control configuration of a pulse wave measuring apparatus of the first embodiment.

FIG. 7A is a diagram illustrating an acceleration pulse wave.

FIGS. 7B to 7D are diagrams illustrating a method for estimating blood vessel age based on the acceleration pulse wave.

FIGS. 8A to 8D are diagrams showing a pulse wave measuring apparatus of a wrist-wrapping type according to a third embodiment.

### DESCRIPTION OF THE EMBODIMENTS

Hereinafter, embodiments of the present invention will be described with reference to the accompanying drawings.

### First Embodiment

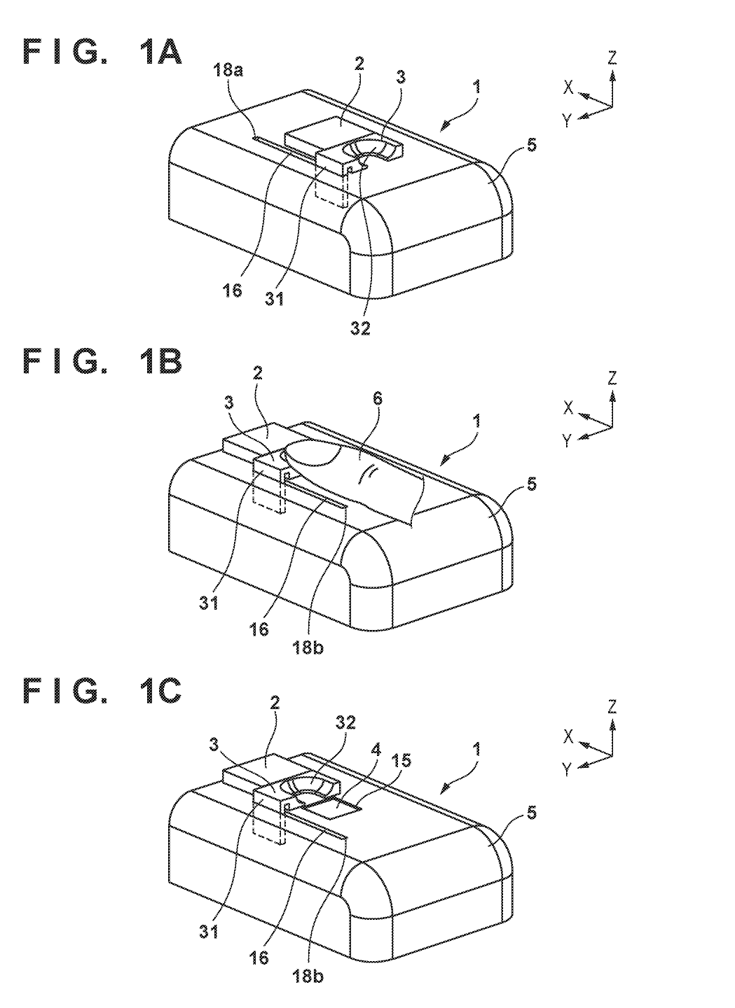

FIGS. 1A to 1C are external perspective views showing a pulse wave measuring apparatus 1 serving as a biological information measurement apparatus according to a first embodiment. The pulse wave measuring apparatus 1 has a housing 5 that contains a spectrometer. The upper surface of the housing 5 is a surface on which a measurement target is to be placed. The upper surface of the housing 5 is provided with an aperture portion 15 that enables the coming and going of light between a measurement target placed on the upper surface and the spectrometer inside of the housing 5, and a transparent cover 4 composed of a transparent material that covers the aperture portion 15. Also, the upper portion of the aperture portion 15 and the transparent cover 4 is provided with a shutter member 2, and a guide member 3 for guiding the measurement target. Note that FIG. 1A is a diagram showing a state in which the shutter member 2 covers the aperture portion 15, FIG. 1B is a diagram showing a state in which the shutter member 2 has retreated, the aperture portion 15 is open, and a finger 6, which is the measurement target, covers the aperture portion 15, and FIG. 1C is a diagram in which the illustration of the finger is omitted in the state shown in FIG. 1B.

In the present embodiment, the shutter member 2 and the guide member 3 are connected, or are constituted integrally. The guide member 3 has a guide shape portion 31 and a finger receiving portion 32. The guide member 3 and the shutter member 2 can perform a sliding movement in the X direction shown in FIGS. 1A to 1C due to the guide shape portion 31 and a guide rail portion 16 provided on the housing 5. The two end portions of the guide rail portion 16 function as a stopper portion 18a and a stopper portion 18b and define two positions, namely a position at which the guide shape portion 31 abuts against the stopper portion 18a, and a position at which the guide shape portion 31 abuts against the stopper portion 18b. Due to the guide member 3 being moved by the finger 6, the shutter member 2 can move between a first position (FIG. 1A) of covering the aperture portion 15 at the position opposing the aperture portion 15 of the housing 5, and a second position (FIG. 1C) of retreating from the position opposing the aperture portion 15.

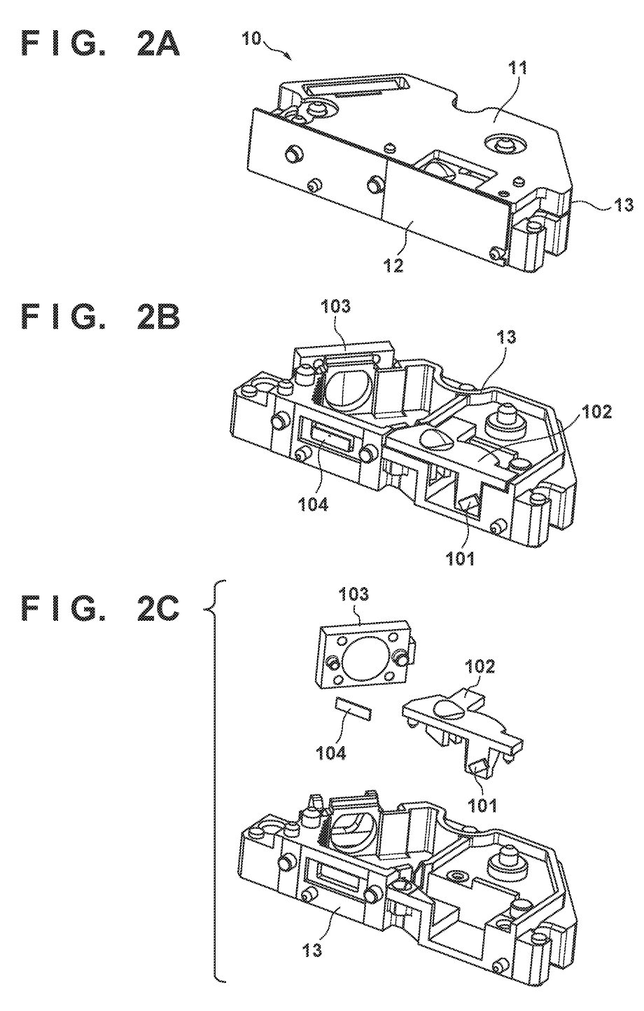

FIG. 2A shows the appearance of a spectrometer 10 contained and arranged in the housing 5 of the pulse wave measuring apparatus 1. An outer shell of the spectrometer 10 is formed by a cover member 11 and a case member 13. An electric substrate 12 has a circuit or the like for amplifying the signal from the line sensor 104, performing A/D conversion on the resulting signal, and thus obtaining an output signal (digital signal) for each wavelength. FIG. 2B shows a state in which the cover member 11 and the electric substrate 12 of the spectrometer 10 have been removed. FIG. 2C shows a configuration in which an optical system and a light receiving sensor are divided and extracted to the upper portion from the case member 13.

The optical system of the spectrometer 10 includes: a white LED 101, which is a light source for illuminating the measurement target; a light guide 102; a diffraction grating 103; and the line sensor 104. The light guide 102 is a light guiding member in which an illuminating portion for guiding a luminous flux from the white LED 101 serving as a white light source to the measurement target, and a light collecting portion for collecting and guiding the reflected light from the measurement target are integrated. The light from the white LED 101 is guided to the aperture portion 15 by the illuminating portion of the light guide 102, passes through the aperture portion 15, and illuminates the measurement target (in this example, the finger 6). The reflected light from the measurement target is collected and guided by the light guiding portion of the light guide 102, and is guided to the diffraction grating 103, which is a spectral unit that disperses light with a predetermined resolution in a predetermined wavelength range. The reflected light is dispersed into multiple wavelengths by the diffraction grating 103 serving as a spectral unit, and a line sensor 104 serving as a light reception portion receives the dispersed light. Light receiving elements that receive light resolved into multiple wavelengths are arranged in parallel in the line sensor 104. In the spectrometer 10, the white LED 101, the light guide 102, the diffraction grating 103, and the line sensor 104 are constituted integrally, and thus a reduction in size is realized.

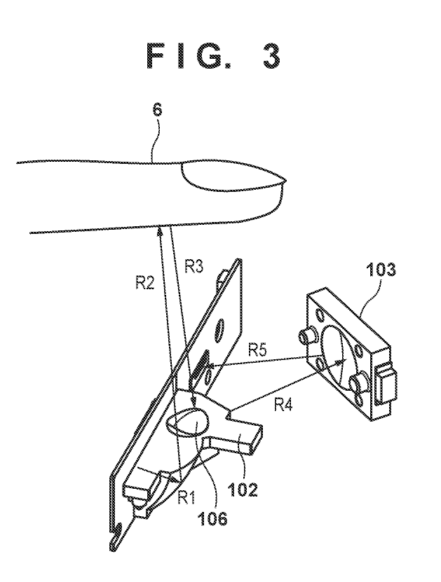

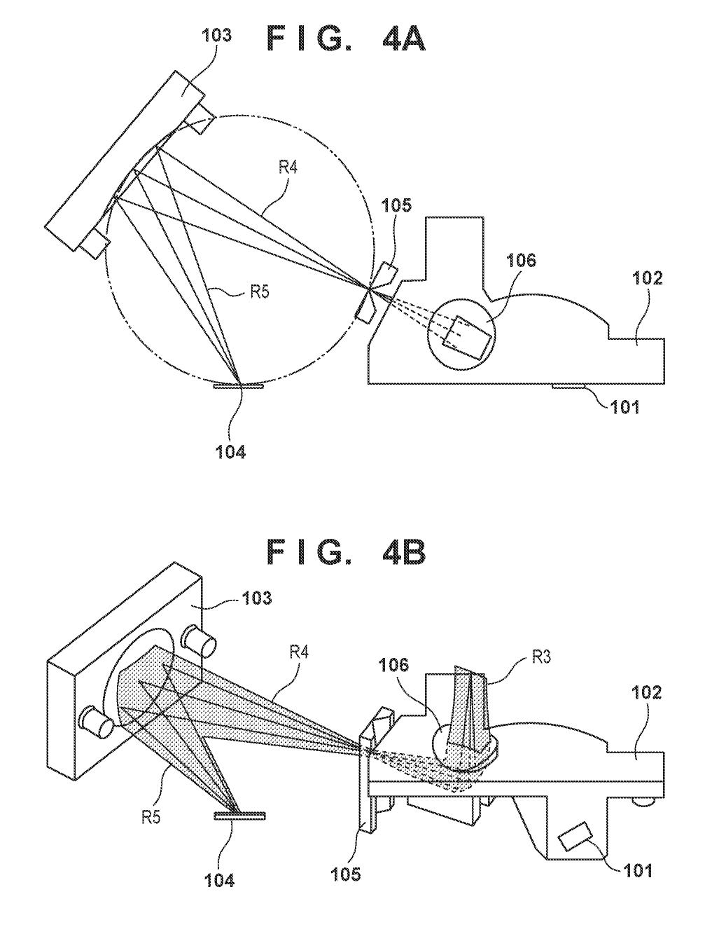

FIG. 3 is a diagram in which the configuration of the optical system in the spectrometer 10 is extracted and the advancing state of the light rays emitted from the white LED 101 is indicated by the arrows R1 to R5. Also, FIG. 4A shows a top view of the optical system showing a dispersed state of the reflected light, and FIG. 4B shows a perspective view of the optical system showing a dispersed state of the reflected light.

The luminous flux R1 emitted from the white LED 101 installed on the electric substrate 12 of the spectrometer 10 is reflected by the curved surface portion of the light guide 102 obtained through resin molding, and is emitted as illuminating light R2 to the upper surface. The illuminating light R2 passes through the aperture portion 15 and the transparent cover 4, and illuminates the measurement target (in the present embodiment, the abdominal portion of the finger 6) of the living body placed on the transparent cover 4. The reflected light R3 from the illuminated region is incident on an incidence portion 106 of the light guide 102, which is obtained through resin molding.

The reflected light that is incident on the incidence portion 106 is collected and guided by the light guide 102 and is used as a luminous flux R4 to illuminate the diffraction grating 103 via a slit portion 105 with a minute width. The slit portion 105 is contained in and fixed to the case member 13. The diffraction grating 103 is produced using resin, and is a concave reflection type of diffraction grating (concave diffraction grating) obtained by forming a diffraction grating on a concave surface. The diffraction grating 103 is created by vapor-depositing a reflection film, comprising aluminum or the like, or an enhanced reflection film, comprising SiO₂ or the like, on the diffraction grating surface. The luminous flux R5 dispersed by such a diffraction grating 103 is used to illuminate the line sensor 104 installed on the electric substrate 12. In the present embodiment, the light source (white LED 101) and the light receiving portion (line sensor 104) are arranged on the electric substrate 12, that is, on the same substrate.

As described above, the line sensor 104 includes a structure in which multiple light receiving elements that realize photoelectric conversion are arranged linearly (in series). The dispersed lights illuminate the multiple light receiving elements, whereby the light intensity for each wavelength can be measured. For example, if the wavelength resolution is set to 5 nm and 100 light receiving elements are arranged in series in the line sensor 104, the line sensor 104 can capture a wavelength range of about 500 nm. If the shortest wavelength portion to be dispersed is set as 400 nm, for example, the wavelength range is a range that covers from the visible light region to the near infrared region, or 400 to 900 nm, and the wavelength band that is needed for light measurement can be mostly ensured along with the sensor sensitivity. In the present embodiment, the diffraction grating 103, the line sensor 104, and the slit portion 105 are arranged on the circumference of a so-called Rowland circle, which is indicated by the two-dot chain line in FIG. 4A, and thereby a small spectrometer that disperses the reflected light from the measurement target into predetermined light wavelengths is realized.

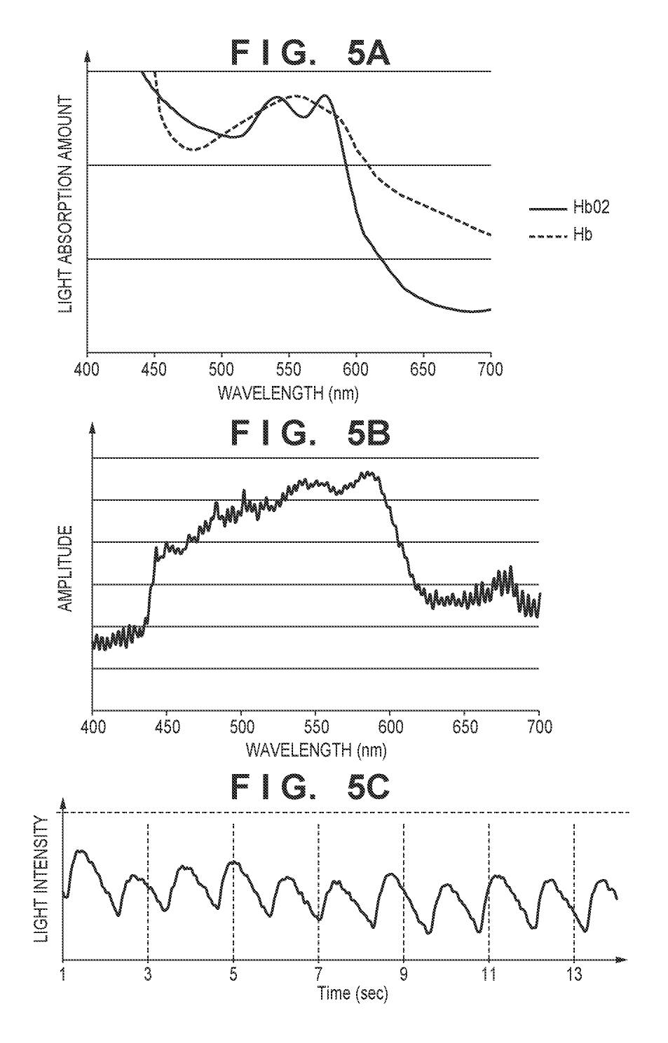

It is commonly known that if the fingertip is illuminated with the luminous flux emitted from the spectrometer 10, the pulse of the blood can be measured using the difference in optical absorption properties of the oxidized hemoglobin and the reduced hemoglobin in the blood. FIG. 5A is a diagram showing optical absorption properties of oxidized hemoglobin (HbO2) and reduced hemoglobin (Hb). In FIG. 5A, the horizontal axis indicates the light wavelength and the vertical axis indicates the light absorption amount.

FIG. 5B is a diagram showing the amplitudes of a pulse wave measured by the spectrometer 10 for wavelengths in the visible light region of 400 to 700 nm by illuminating the fingertip portion with the white LED 101. The horizontal axis indicates the wavelengths, and the vertical axis indicates the averaged amplitude amounts of the wavelengths. The measurement results shown in FIG. 5B are obtained by performing measurement under the following conditions. That is, the line sensor 104 measures the light intensity with a resolution in units of several nanometers in the wavelength range of the entire visible light region, using the above-described visible light region (400 to 700 nm) in the luminous flux dispersed by the diffraction grating 103 as the dispersion band. Note that the light intensities are obtained over multiple instances, and the average value thereof is used as the result of one instance of measuring the light intensity. This is because each signal includes some amount of variation. Hereinafter, the average value is considered the measured light intensity. The light intensity at every several tens of milliseconds is measured over approximately one minute by the line sensor 104 based on the reflected light of the entire wavelength range, and the amplitude (peak to peak) of the variation (pulse wave) of the light intensity for each wavelength is obtained based on the obtained approximately one minute's worth of data. Furthermore, the amplitude of the pulse wave is normalized based on the measurement data of a white reference carried out separately. FIG. 5B shows data obtained by averaging the multiple normalized amplitude values obtained by carrying out the above-described processing multiple times.

In general, an LED of a single color such as green, or the like is used as the light source and the reflected light amount is measured. In this case, the wavelength is around 550 nm. As can be understood from FIG. 5B, a comparatively large amplitude is obtained also around 550 nm, but the largest amplitude is obtained in the wavelength range of 570 to 590 nm, which has the highest absorption level in the visible light region. In view of this, in the present embodiment, the pulse wave is obtained by sequentially extracting the light intensities for the wavelength of 590 nm, which has the largest amplitude. FIG. 5C shows the results of measuring the light intensity for the wavelength of 590 nm over approximately 13 seconds. The horizontal axis indicates time (seconds) and the vertical axis indicates the intensity of the received light amount (light intensity). This wavelength indicates a pulse wave that accompanies so-called movement of the blood in the blood vessel.

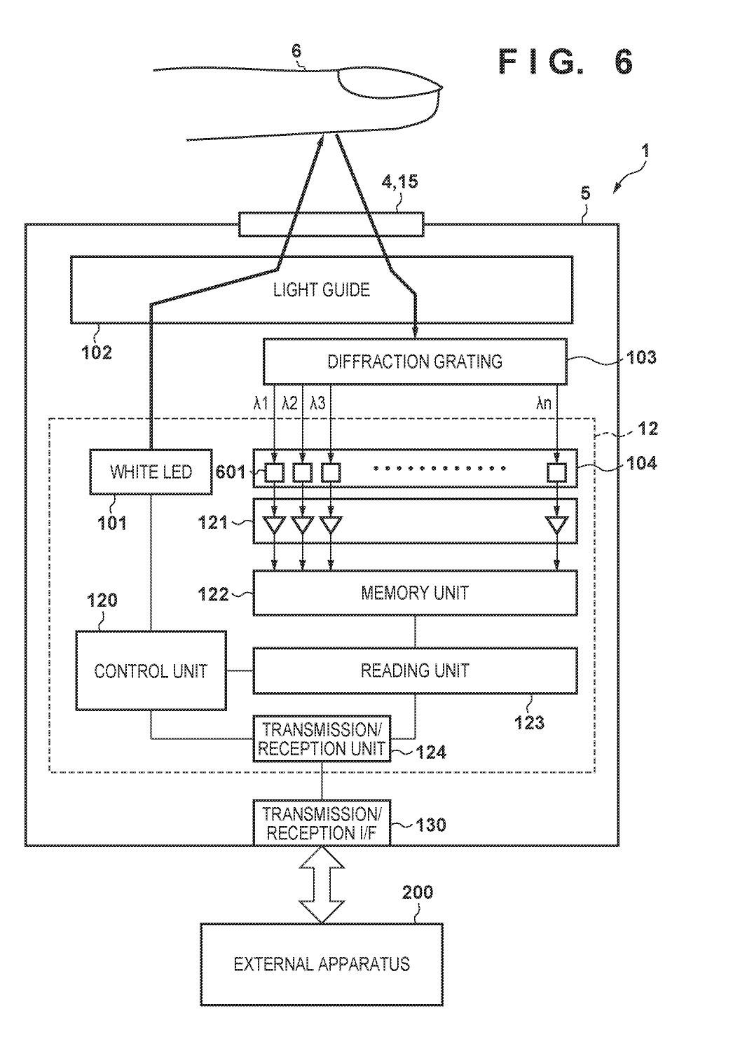

FIG. 6 is a block diagram showing an example of a control configuration of the pulse wave measuring apparatus 1. As described above, the luminous flux from the white LED 101 goes through the light guide 102 and illuminates the measurement target outside of the housing 5 from the transparent cover 4 (aperture portion 15). In the present embodiment, the light from the white LED 101 illuminates the abdominal portion of the tip of the finger 6 arranged at the measurement position (the position of the transparent cover 4) of the pulse wave measuring apparatus 1. The reflected light from the measurement target goes through the transparent cover 4, enters the inside of the housing 5, and is guided to the diffraction grating 103 by the light guide 102. The diffraction grating 103 disperses the reflected light into multiple wavelengths (λ1 to λn) and illuminates the line sensor 104.

The line sensor 104 has multiple light receiving elements 601 for converting the light intensities for the multiple wavelengths from the diffraction grating 103 into electrical signals. The multiple light receiving elements 601 output the electrical signals indicating the light intensities for all dispersed wavelengths. The signal processing unit 121 amplifies the electrical signals output from the multiple light receiving elements 601, subjects them to A/D conversion, and transmits the result to the memory unit 122 as information on the light intensity (digital measurement values). The memory unit 122 temporarily stores the light intensities output from the signal processing unit 121. In this manner, the light intensities for each wavelength obtained by the line sensor 104 are sequentially stored in the memory unit 122.

A reading unit 123 sequentially reads out the light intensities for a predetermined wavelength (in this example, 590 nm) among the light intensities for all wavelengths stored in the memory unit 122, and sends the read-out light intensities to a transmission/reception unit 124. The transmission/reception unit 124 sequentially sends the light intensities for the predetermined wavelength to an external apparatus 200 via a transmission/reception I/F 130. The transmission/reception I/F 130 may use a wire or be wireless. In this manner, the pulse wave data of the specific wavelength can be checked by reading out and displaying the temporal variation in the specific wavelength on a screen of the external apparatus 200, which is a PC or the like. The control unit 120 includes a processor and a memory, and performs communication with the external apparatus 200 and overall control of the above-described units.

Note that the light intensities for the wavelengths in the memory unit 122 are overwritten based on the next piece of chronological data from the signal processing unit 121. Also, it is practical to set the measurement value of one instance of light intensity by averaging the multiple output values from the line sensor 104 as described above. Accordingly, the signal processing unit 121 has a configuration for performing processing for adding and averaging multiple signals from the light receiving element of the line sensor 104.

According to the above-described pulse wave measuring apparatus 1, the luminous flux illuminates the fingertip portion and the pulse wave can be measured based on the reflected light therefrom, using a configuration in which a white LED light source and a spectrometer are used. If the number of wavelength peaks per unit time is detected on the basis of the waveform (pulse wave) obtained as described above, the pulse rate can be detected. It is also possible to estimate the consumed energy amount based on the pulse count per unit time.

If the second-order differentials of these pulse wave waveforms are found formulaically, so-called acceleration pulse waves can be obtained. The pulse wave waveform obtained through common pulse wave measurement using a single-color LED includes light intensities for unneeded wavelengths, and if the second-order differential thereof is found, these light intensities will have a prominent influence. For this reason, it will not be possible to obtain an accurate acceleration pulse wave. In contrast to this, according to the pulse wave measuring apparatus 1 of the present embodiment, the light intensities for the needed wavelength can be obtained using a spectrometer, and therefore an accurate acceleration waveform can be obtained.

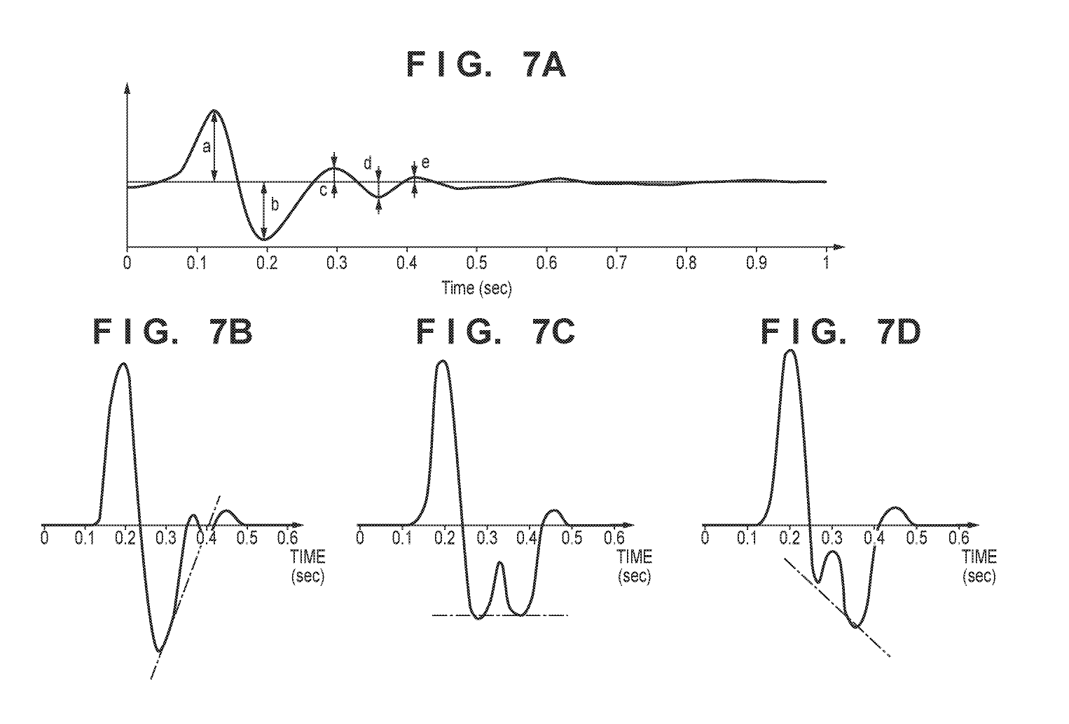

FIG. 7A shows a representative acceleration waveform. Being able to divide and evaluate the degree of aging of the blood vessel in approximately seven stages using the amplitude values of a to e shown in the acceleration pulse wave is a known technique. Specifically, for example, the approximate degree of aging of the blood vessels can be estimated on the basis of the slope of the line segment obtained by connecting b/a and d/a. FIGS. 7B to 7D show an example of this. In the drawings, line segments obtained by connecting a b value and a d value are indicated by two-dot chain lines. The flexibility of the blood vessels can be indicated by the slopes of the line segments connecting the b/a values and the d/a values normalized by the initial amplitude value a of the acceleration pulse wave, and it has been said that these flexibilities approximately correspond also to the age of the person. Note that the drawings show the trends of the slopes of the b/a values and the d/a values by showing the slope from the b value to the d value.

FIG. 7B shows the case of an acceleration waveform obtained with a highly flexible blood vessel of a young person approximately in their 20s or 30s. The b value is large, the d value is comparatively small, and the line segment slopes upward to the right. Also, it has been said that in general, as one ages, the flexibility of the blood vessels decreases, or the degree of blood vessel stiffness increases, and accordingly, the slope of the line segment changes as shown in FIGS. 7C and 7D. The state of sloping downward to the right, as shown in FIG. 7D, is shown as a representative waveform of a person in his or her 60s, and it is known that the b value is small and the d value is large.

Also, the wavelength on the light receiving line sensor is selectively chosen, and therefore unneeded wavelength component light, such as external light, is not taken in, and thus there is a high resistance to noise caused by external light. Accordingly, with the present embodiment, a pulse wave measuring apparatus with a small size and high performance is provided, which realizes highly-accurate pulse wave measurement through use of a spectrometer.

As long as the light intensity for a specific wavelength, that is, the light intensity for a single wavelength, is used, a sensor that detects the light intensity for the specific wavelength in the light dispersed by the diffraction grating 103 may also be used instead of the line sensor 104. For example, the sensor need only have an light receiving element arranged at a position for detecting the light intensity for the wavelength 590 nm and be configured to store the information on the light intensity obtained by the light receiving element in the memory unit 122.

The control unit 120 may also control the reading unit 123 so as to read out the light intensities for the selected wavelength based on the instruction for selecting the wavelength, received from the external apparatus 200 by the transmission/reception unit 124. That is, the transmission/reception unit 124 functions as a reception unit for receiving an instruction for selecting the wavelength from the external apparatus 200, and the control unit 120 controls the reading unit 123 so as to sequentially obtain the light intensities for the wavelength selected using the instruction received by the reception unit. If this kind of configuration is used, the light intensity for any wavelength can be used by the instruction from the external apparatus 200.

Furthermore, the reading unit 123 may also read out the light intensities for multiple wavelengths from the memory unit 122. If the pulse wave measuring apparatus 1 using the spectrometer 10 is used, the illuminating light on the measurement target is the light of a single light source, and therefore a signal with low noise can be obtained even if multiple wavelengths are selected. An example of a biological information measuring apparatus for measuring the light intensities for multiple wavelengths will be described with reference to a second embodiment below.

### Second Embodiment

A pulse wave measuring apparatus serving as a biological information measurement apparatus according to a second embodiment will be described. In the first embodiment, a configuration was described in which a pulse wave is measured using light intensities for one wavelength as the biological information. In the second embodiment, variation in the light intensities for multiple wavelengths is obtained. In general, in the case of using multiple different wavelengths, multiple light sources corresponding to the respective wavelengths are arranged, and therefore the position of the light source differs for each wavelength. As a result, regardless of the fact that it is desirable to illuminate the measurement target at the same location, the illumination positions are shifted relative to each other because the multiple light sources are arranged at different positions, which causes noise in the light reception signal. Also, if an attempt is made to simultaneously install the multiple light sources, a problem also occurs in that the installation area of the light sources becomes excessively large with respect to the illumination target portion, and thus the illumination portion of the apparatus, and by extension the entire apparatus, becomes too large.

A pulse oscillometer for measuring the blood oxygen saturation concentration exists as an example of measuring biological information using light of multiple wavelengths. With the pulse oscillometer, the light of the multiple wavelengths (red and near-infrared) illuminates and passes through the fingertip portion or the like, and is received by a light receiving sensor. Based on the received light amount, the blood oxygen saturation concentration is calculated using different light absorption ratios for the wavelengths of oxidized hemoglobin and reduced hemoglobin. Specifically, the pulse oscillometer causes the red light (wavelength near 660 nm) and the near-infrared light (near 940 nm) to pass through the fingertip portion, an ear lobe, or the like, and estimates the blood oxygen saturation concentration (SpO2) based on the variation in the light intensity at that time. The hemoglobin that exists in large quantities in the blood is oxidized hemoglobin and reduced hemoglobin. If the red-colored light is allowed to pass through, the absorption level of the reduced hemoglobin is greater than the absorption level of the oxidized hemoglobin, and in the case of the near-infrared light, the reduced hemoglobin has a slightly lower optical absorption property than the oxidized hemoglobin. For this reason, the ratio of the light absorption levels of the red and near-infrared light of the hemoglobin can be said to change depending on the oxygen saturation level, which is the ratio between “oxidized hemoglobin” and “oxidized hemoglobin+reduced hemoglobin”.

Here, a light absorption level change αA caused by the pulse wave can be expressed as a ratio, αI/I, between the intensity change αI and the transmitted light intensity I of the transmitted light caused by the pulse wave, by applying the Beer-Lambert law. This value can be replaced with a ratio AC/DC between the variation component AC component of the pulse wave to be measured and the DC component indicated by the absorption and the like of venous blood and the like.

Here, letting R be the ratio of the oxygen saturation levels,

R=red light/infrared light=(AC660/DC660)/(AC940/DC940) Equation (1)

where 660 and 940 indicate wavelengths.

Furthermore, this R value can be applied to a calibration curve of an R value and SpO2 obtained in advance with the wavelength (660 nm) of the red light and the wavelength (940 nm) of the near-infrared light, whereby the blood oxygen saturation concentration SpO2 can be obtained.

A transmitting type was described above as a prerequisite, but in principle, similar measurement is possible also with a reflecting type.

In actual measurement, error occurs in some cases in the calibration curve since the wavelengths of the red or near-infrared LEDs constituting these apparatuses are somewhat shifted from each other. Also, correction of the emitted light amount of the LED, the sensitivity of the light receiving sensor, and the like is necessary depending on the thickness of the skin at the measurement site, the color of the skin, and the like. There are also cases in which the amount by which the influence of respiration, bodily movement, and the like varies during measurement cannot be ignored. In order to cancel out these influences, for example, Japanese Patent Laid-Open No. 2005-095606 has proposed measuring light intensities for five wavelengths. In the case of measuring the light intensities for five wavelengths, there is a risk that the pulse wave measuring apparatus will increase in size if multiple light sources corresponding to the multiple wavelengths are used.

In the second embodiment, the wavelength band of the illuminating light of the white LED is ensured, and the wavelength region to be dispersed and detected is set to a region of about 600 nm to 1000 nm, for example. The reading unit 123 reads out the light amount values of the multiple wavelengths (if red and near-infrared are used, the wavelengths 660 nm and 940 nm) from the memory unit 122 and transmits the read-out light amount values to the external apparatus 200 via the transmission/reception unit 124. The external apparatus 200 can realize the function of the pulse oscillometer based on the light amount variation over time.

Also, as described above, the influences of the respiration and bodily movement can be canceled out by setting five wavelengths (e.g., 660, 700, 730, 805, and 875 nm). These multiple wavelengths can be realized through illumination of a target portion using a white LED as a single light source, and can be selected from the wavelengths allocated to the line sensor 104 of the spectrometer 10. That is, according to the biological information measuring apparatus 1 of the second embodiment, which has the spectrometer 10, measurement of the light intensities for the above-described five wavelengths can be realized easily due to the reading unit 123 being configured to read out the light intensity information for the five wavelengths from the memory unit 122. Accordingly, there is no need to install a light source for each individual illuminating wavelength, and an increase in the size of the apparatus can be avoided. That is, it is possible to provide a small-sized and highly-accurate pulse oscillometer.

Furthermore, with a two-wavelength pulse oscillometer, the optical absorption properties of carboxyhemoglobin COHb and oxidized hemoglobin HbO2 approximately match at the set wavelength 660 nm. For this reason, carboxyhemoglobin COHb and oxidized hemoglobin HbO2 cannot be recognized as being distinct, and there is a risk that a patient will be mistaken for being in a state of carbon monoxide poisoning. Also, it is known that a rise in the carbon monoxide concentration accompanies septicemia, and it is thought that it is important to monitor the CO concentration. In view of this, by selecting seven or more types of wavelengths, various types of hemoglobin (oxidized hemoglobin, reduced hemoglobin, carboxyhemoglobin, methemoglobin) can be identified and the influence of bodily movement can be canceled out. This example has been introduced by Masimo Corporation (Masimo Rainbow® SET pulse CO oximetry).

Even in a state such as that described above in which light intensities for seven wavelengths are measured simultaneously, the reading unit 123 can selectively read out the wavelengths from the memory unit 122 as long as they are wavelengths in a wavelength range in which the wavelengths can be detected by the line sensor 104. Accordingly, with the present embodiment, it is possible to provide an apparatus that can detect a pulse wave, a carboxyhemoglobin concentration, and a carbon monoxide concentration while canceling out the influence of bodily movement.

Also, since a single light source is used, no shifting in the illumination position occurs as in the case where multiple light sources are arranged, and a reduction of noise in the light reception signal is realized. Also, due to the fact that substantial variation and the like in the set light source wavelength is also a wavelength component obtained by dispersing the same light source, it is possible to reduce error. Also, since the multiple dispersed wavelengths can be selected as appropriate, the optimal wavelength and combination of wavelengths can be selected according to the measurement target, and therefore reduction of various types of noise can be realized.

Note that the control unit 120 may also perform calculation using the above-described external apparatus 200. Also, a display unit for displaying the results of this calculation may also be provided in the pulse wave measuring apparatus 1.

### Third Embodiment

Next, a pulse wave measuring apparatus according to a third embodiment will be described. By employing the small-sized spectrometer 10, it is also possible to provide a wearable pulse wave measuring apparatus 1, for example, in addition to a pulse wave measuring apparatus that performs measurement on a desktop. In the third embodiment, a pulse wave measurement apparatus in a mode of being worn on an arm will be described.

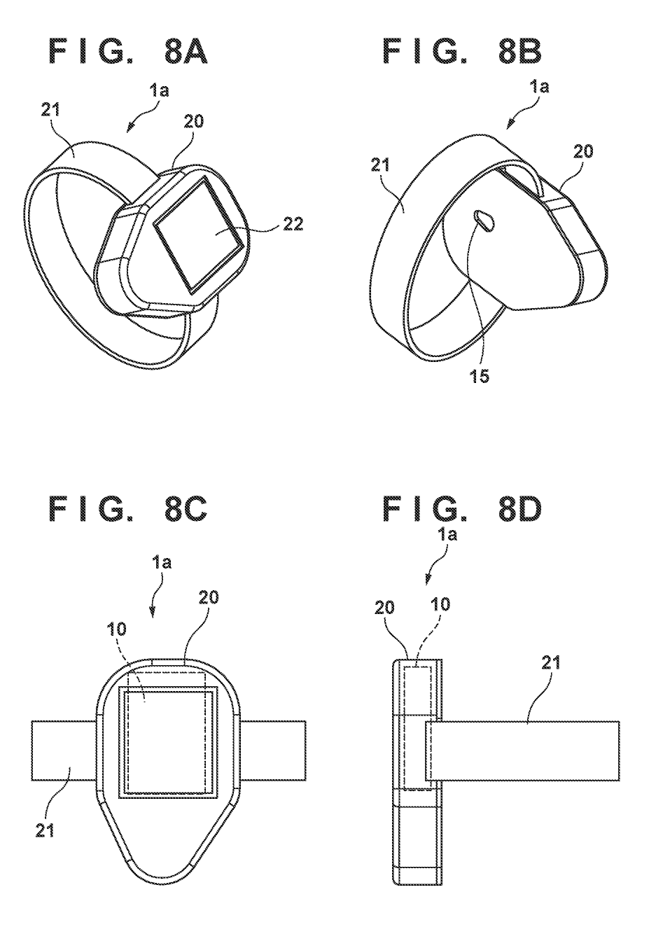

As shown in FIG. 8A, a pulse wave measurement apparatus la serving as a biological information measuring apparatus of the third embodiment includes a main body portion 20 and a band 21 for wearing the main body portion 20 on an arm portion, and the pulse wave measuring apparatus includes a monitor portion 22 for displaying measurement results and the like on a surface of the main body portion 20. FIGS. 8C and 8D show a main body front view and a side view respectively. The spectrometer 10, which is indicated by a broken line, is provided inside of the main body portion 20. The spectrometer 10 has the configuration described in the first and second embodiments. Also, FIG. 8B shows a perspective view of the apparatus in the case where the main body portion 20 is viewed from the rear surface. An aperture portion 15 for illuminating with light is installed at part of the rear surface region of the main body portion 20, which is worn on an arm.

When the pulse wave measuring apparatus la is instructed to start measurement in a state in which it is worn on the arm portion, the pulse wave measuring apparatus la intermittently illuminates the arm portion surface with light from the aperture portion 15. Then, the pulse wave measuring apparatus la takes the reflected light into the apparatus via the aperture portion 15, disperses the light using the spectrometer 10, detects the light intensity for a desired wavelength, and can evaluate the pulse wave, for example.

As described above, according to the third embodiment, long-term pulse wave measurement in the state of being worn on the arm, pulse wave measurement during sleep, and the like can be realized easily. Also, the burden of being aware of a measurement examination is reduced for the wearer as well.

As described above, according to the first to third embodiments, by forming a biological information measuring apparatus using a spectrometer 10 with a reduced size, a biological information measuring apparatus is provided which can stably detect a pulse wave or the like in a state of being installed on a desktop or on an arm or the like. Also, by realizing a smaller size, it is possible to reduce the area of the space occupied when the biological information measuring apparatus is installed on a desktop. Furthermore, if the biological information measuring apparatus can be installed on a wrist or the like of the measurement subject, the degree of freedom in the measurement, such as being able to measure the biological information while the measurement subject is moving, can be increased.

As described above, according to the above-described embodiments, by applying a spectrometer to the detection of biological information, it is possible to commercially provide a small-size biological information measurement apparatus that can stably detect, with high accuracy, biological information such as a pulse wave, with an inexpensive configuration.

It should be noted that in the above-described first to third embodiments, the light intensities of the light of multiple wavelengths (λ1 to λ3) obtained from the diffraction grating 103 are stored in the memory unit 122, and the reading unit 123 reads out the light intensities for a predetermined wavelength to be used for measurement from the memory unit 122. That is, the reading unit 123 sequentially reads out the light intensities for a predetermined wavelength, which are part of the light intensities for the multiple wavelengths stored in the memory unit 122, but there is no limitation to this. For example, the light intensities of the light of the predetermined wavelength to be used for measurement, which is part of the light of the multiple wavelengths obtained from the diffraction grating 103, may be detected and stored in the memory 122, and the reading unit 123 may sequentially read out the light intensities from the memory unit 122. By doing so, the number of light receiving elements 601 in the line sensor 104 can be reduced.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2018-060730, filed Mar. 27, 2018, which is hereby incorporated by reference herein in its entirety.

What is claimed is:

1. A biological information measuring apparatus, comprising:
a light source;
a guide unit configured to guide a luminous flux from the light source to a measurement target, which is part of a living body, and to guide reflected light from the measurement target;
a spectral unit configured to disperse the reflected light guided by the guide unit, with a predetermined resolution in a predetermined wavelength range; and
an obtaining unit configured to sequentially obtain light intensities for a predetermined wavelength, which is part of light of a plurality of wavelengths obtained by the spectral unit.

2. The apparatus according to claim 1, wherein
the obtaining unit includes a detecting unit configured to detect light intensities for a predetermined wavelength, which is part of the light of the plurality of wavelengths obtained by the spectral unit, and
the obtaining unit sequentially obtains the light intensities for the wavelength detected by the detecting unit.

3. The apparatus according to claim 1, wherein
the obtaining unit includes a detecting unit configured to detect light intensities for at least one wavelength of the light of the plurality of wavelengths obtained by the spectral unit, and
the obtaining unit sequentially obtains light intensities for a predetermined wavelength, which are part of the light intensities for the wavelengths detected by the detecting unit.

4. The apparatus according to claim 3, wherein
the detecting unit includes a plurality of light receiving elements configured to detect the light intensities for the plurality of wavelengths, and stores information indicating the light intensities obtained from the plurality of light receiving elements in a memory unit, and
the obtaining unit sequentially reads out the light intensities for the predetermined wavelength from the memory unit.

5. The apparatus according to claim 4, wherein the obtaining unit obtains light intensities for a plurality of predetermined wavelengths from the memory unit.

6. The apparatus according to claim 1, wherein the guide unit includes a guide member in which an illuminating portion configured to guide the luminous flux to the measurement target and a light collection portion configured to collect and guide the reflected light are integrated.

7. The apparatus according to claim 6, wherein the spectral unit includes a diffraction grating configured to disperse the reflected light collected and guided by the guide member.

8. The apparatus according to claim 7, wherein the detecting unit includes a plurality of light receiving elements arranged in series in order to detect light intensities of a luminous flux dispersed by the diffraction grating, for each wavelength.

9. The apparatus according to claim 7, wherein the diffraction grating is a concave diffraction grating.

10. The apparatus according to claim 1, wherein the light source, the guide unit, and the detecting unit are integrated by a case member.

11. The apparatus according to claim 1, comprising a reception unit configured to receive an instruction for selecting a wavelength from an external apparatus,
wherein the obtaining unit sequentially obtains light intensities for a wavelength selected using the instruction received by the reception unit.

12. The apparatus according to claim 1, wherein the light source is a white light source.

13. The apparatus according to claim 1, wherein the light source and the detecting unit are arranged on the same substrate.

14. The apparatus according to claim 1, wherein the predetermined wavelength range includes regions from a visible light region to a near-infrared region.

15. The apparatus according to claim 1, wherein the predetermined wavelength range is a visible light region.

16. A biological information measurement method performed by a biological information measurement apparatus, the method comprising:
guiding a luminous flux from a light source to a measurement target, which is part of a living body;
dispersing reflected light from the measurement target with a predetermined resolution in a predetermined wavelength range by guiding the reflected light to a spectral unit; and
sequentially obtaining light intensities for a predetermined wavelength, which is part of light of a plurality of wavelengths obtained through the dispersion performed by the spectral unit.
